(19) United States
(12) Patent Application Publication
(10) Pub. No.: US 2008/0009722 A1
(43) Pub. Date: Jan. 10, 2008

(54) Multi-planar reconstruction for ultrasound volume data

(76) Inventors: Constantine Simopoulos, San Francisco, CA (US); Lewis J. Thomas, Palo Alto, CA (US); Shaohua Kevin Zhou, Plainsboro, NJ (US); Dorin Comaniciu, Princeton Junction, NJ (US); Bogdan Georgescu, Plainsboro, NJ (US)

Correspondence Address: SIEMENS CORPORATION;INTELLECTUAL PROPERTY DEPARTMENT, 170 WOOD AVENUE SOUTH, ISELIN, NJ, 08830, US

(21) Appl. No.: 11/527,286

(22) Filed: Sep. 25, 2006

Related U.S. Application Data
(60) Provisional application No. 60/747,024, filed on May 11, 2006.

(51) Int. Cl. A61B 8/00

(52) U.S. Cl. 600/437

(57) ABSTRACT

During scanning or in real-time with acquisition of ultrasound data, a plurality of images is generated corresponding to a plurality of different planes in a volume. The volume scan data is searched by a processor to identify desired views. Multiple standard or predetermined views are generated based on plane positioning within the volume by the processor. Multi-planar reconstruction, guided by the processor, allows for real-time imaging of multiple views at a substantially same time. The images corresponding to the identified views are generated independent of the position of the transducer. The planes may be positioned in real-time using a pyramid data structure of coarse and fine data sets.

### RELATED APPLICATIONS

The present patent document claims the benefit of the filing date under 35 U.S.C. §119(e) of Provisional U.S. Patent Application Ser. No. 60/747,024, filed May 11, 2006, which is hereby incorporated by reference.

### BACKGROUND

The present embodiments relate to medical diagnostic ultrasound imaging. In particular, multi-planar reconstruction for ultrasound volume data is provided.

Ultrasound may be used to scan a patient. For example, echocardiography is a commonly used imaging modality to visualize the structure of the heart. Because the echo is often a 2D projection of the 3D human heart, standard views are captured to better visualize the cardiac structures. For example, in the apical four-chamber (A4C) view, all four cavities, namely left and right ventricles, and left and right atria, are present. In the apical two-chamber (A2C) view, only the left ventricle and the left atrium are present. Another example is imaging the intracranial structures of a fetus. Three standard planes are acquired with different orientations, not necessarily orthogonal, but fixed with respect to each other, for visualization of the cerebellum, the cistema magna and lateral ventricles.

Acquired cardiac or other desired views often deviate from the standard views due to machine properties, the inter-patient variations, or preferences of sonographers. The sonographer manually adjusts imaging parameters of the ultrasound system and transducer position, resulting in variation. For example, the user moves the imaging plane and associated view by moving the transducer relative to the patient. Undesired movement by the patient and/or the sonographer may result in an undesired or non-optimal view for diagnosis. U.S. Published Patent Application No. 2005/0096538 discloses stabilizing the view plane relative to the patient despite some transducer movement. The user positions the plane to a desired location in the patient. Subsequently, the scan plane is varied relative to the transducer to maintain the scan plane in the desired location relative to the patient.

Real-time 3D ultrasound, such as in echocardiography, is an emerging technique that visualizes a volume region of the patient, such as the human heart in spatial and temporal dimensions. Multiple images may be obtained at a substantially same time. In multi-planar reconstruction, a volume region is scanned. Rather than or in addition to three-dimensional rendering, a plurality of planes, such as three planes at substantially right angles to each other, are positioned relative to the volume. Two-dimensional images are generated for each of the planes. However, the views of interest may have different positions relative to each other in the volume. If one plane is aligned to the desired view, other planes may not be aligned. For real-time scanning, the planes are defined relative to the transducer. Movement of the transducer relative to the patient may result in non-optimal views. Movement of the object of interest, such as fetus, may result in non-optimal views or constant adjustment of the transducer by the sonographer.

### BRIEF SUMMARY

By way of introduction, the preferred embodiments described below include methods, computer readable media and systems for multi-planar reconstruction for ultrasound volume data. During scanning or in real-time with acquisition of ultrasound data, a plurality of images is generated corresponding to a plurality of different planes in a volume. The volume scan data is searched by a processor to identify desired views. Multiple standard or predetermined views are generated based on plane positioning within the volume by the processor. Multi-planar reconstruction, guided by the processor, allows for real-time imaging of multiple views at a substantially same time. The images corresponding to the identified views are generated independent of the position of the transducer. In a same or other embodiment, the planes are positioned in real-time using a pyramid data structure of coarse and fine data sets.

In a first aspect, a method is provided for multi-planar reconstruction for ultrasound volume data. An ultrasound transducer is positioned adjacent, on or within a patient. A volume region is scanned with the ultrasound transducer. A processor determines, from data responsive to the scanning, a first orientation of an object within the volume region while scanning. A multi-planar reconstruction is oriented as a function of the first orientation of the object and independently of a second orientation of the ultrasound transducer relative to the object. Multi-planar reconstruction images of the object are generated from the data while scanning. The images are a function of the orientation of the multi-planar reconstruction.

In a second aspect, a computer readable storage medium has stored therein data representing instructions executable by a programmed processor for multi-planar reconstruction from ultrasound volume data. The storage medium includes instructions for controlling acquisition by scanning a volume region of a patient; determining, from ultrasound data responsive to the acquisition, locations of features of an object within the volume region represented by the data, the determining being during control of the acquisition by scanning; orienting a plurality of planes within the volume region as a function of the locations of the features, the orienting being independent of an orientation of an ultrasound transducer relative to the object, each of the plurality of planes being different from the other ones of the plurality of planes; and generating images of the object from the data for each of the planes.

In a third aspect, a method is provided for multi-planar reconstruction from ultrasound volume data. Ultrasound data in a first coarse set and a second fine set is obtained. The ultrasound data represents an object in a volume. A processor identifies a plurality of features of the object from the first coarse set of ultrasound data. The processor determines locations of planes for multi-planar reconstruction as a function of the features of the object. The processor refines the locations as a function of the second fine set.

The present invention is defined by the following claims, and nothing in this section should be taken as a limitation on those claims. Further aspects and advantages of the invention are discussed below in conjunction with the preferred embodiments and may be later claimed independently or in combination.

### BRIEF DESCRIPTION OF THE DRAWINGS

The components and the figures are not necessarily to scale, emphasis instead being placed upon illustrating the principles of the invention. Moreover, in the figures, like reference numerals designate corresponding parts throughout the different views.

FIG. 1 is a block diagram of one embodiment of a medical ultrasound imaging system;

FIG. 2 is a flow chart diagram of embodiments of methods for multi-planar reconstruction from ultrasound volume data;

FIG. 3 is a graphical representation of a volume region, object and associated planes of a multi-planar reconstruction in one embodiment;

FIG. 4 is a graphical representation of directional filters in various embodiments;

FIG. 5 is a graphical representation of one embodiment of a pyramid data structure; and

FIG. 6 is a graphical representation of one embodiment of an apical four-chamber view.

### DETAILED DESCRIPTION OF THE DRAWINGS AND PRESENTLY PREFERRED EMBODIMENTS

Online or real-time substantially continuous display of different specific anatomical planes may be provided regardless of the orientation of the transducer. A volume is scanned. The data representing the volume is searched for the location of anatomical features associated with planar positions for desired views, such as standard views. A multi-planar reconstruction is provided without the user having to adjust or initially locate a desired view. Multiple views are acquired substantially simultaneously. Since the planes are positioned relative to acquired data and independent of the volume scanning transducer, desired images are generated even where the transducer or imaged object (e.g., heart or fetus) moves. Inexact alignment of the transducer to all of standard planes may be allowed, even for an initial transducer position. Sonographer workflow and acquisition of desired views may be improved.

In an echocardiography example, canonical slice(s) or planes, such as apical four chamber (A4C) and apical two-chamber (A2C) views, are extracted from the data representing a volume. These anatomical planes are continuously displayed irrespective of the orientation of the transducer used in the acquisition of the volume ultrasound data. Visualization of the acquired volumetric data may be simplified while scanning, possibly improving workflow.

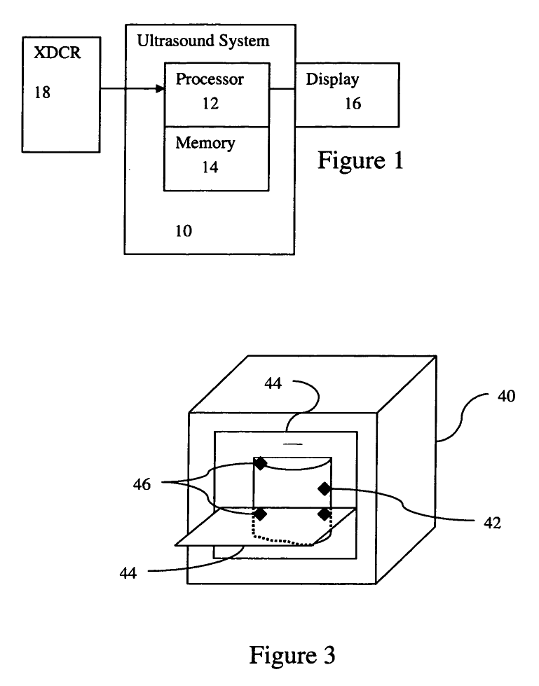

FIG. 1 shows a medical diagnostic imaging system 10 for multi-planar reconstruction from ultrasound volume data. The system 10 is a medical diagnostic ultrasound imaging system, but may be a computer, workstation, database, server, or other system.

The system 10 includes a processor 12, a memory 14, a display 16, and a transducer 18. Additional, different, or fewer components may be provided. For example, the system 10 includes a transmit beamformer, receive beamformer, B-mode detector, Doppler detector, harmonic response detector, contrast agent detector, scan converter, filter, combinations thereof, or other now known or later developed medical diagnostic ultrasound system components.

The transducer 18 is a piezoelectric or capacitive device operable to convert between acoustic and electrical energy. The transducer 18 is an array of elements, such as a multi-dimensional or two-dimensional array. Alternatively, the transducer 18 is a wobbler for mechanical scanning in one dimension and electrical scanning in another dimension.

The system 10 uses the transducer 18 to scan a volume. Electrical and/or mechanical steering allows transmission and reception along different scan lines in the volume. Any scan pattern may be used. In one embodiment, the transmit beam is wide enough for reception along a plurality of scan lines. In another embodiment, a plane, collimated or diverging transmit waveform is provided for reception along a plurality, large number, or all scan lines.

Ultrasound data representing a volume is provided in response to the scanning. The ultrasound data is beamformed, detected, and/or scan converted. The ultrasound data may be in any format, such as polar coordinate, Cartesian coordinate, a three-dimensional grid, two-dimensional planes in Cartesian coordinate with polar coordinate spacing between planes, or other format.

The memory 14 is a buffer, cache, RAM, removable media, hard drive, magnetic, optical, or other now known or later developed memory. The memory 14 is a single device or group of two or more devices. The memory 14 is shown within the system 10, but may be outside or remote from other components of the system 10.

The memory 14 stores the ultrasound data. For example, the memory 14 stores flow (e.g., velocity, energy or both) and/or B-mode ultrasound data. Alternatively, the medical image data is transferred to the processor 12 from another device. The medical image data is a three-dimensional data set, or a sequence of such sets. For example, a sequence of sets over a portion, one, or more heart cycles of the heart are stored. A plurality of sets may be provided, such as associated with imaging a same person, organ or region from different angles or locations.

For real-time imaging, the ultrasound data bypasses the memory 14, is temporarily stored in the memory 14, or is loaded from the memory 14. Real-time imaging may allow delay of a fraction of seconds, or even seconds, between acquisition of data and imaging. For example, real-time imaging is provided by generating the images substantially simultaneously with the acquisition of the data by scanning. While scanning to acquire a next or subsequent set of data, images are generated for a previous set of data. The imaging occurs during the same imaging session used to acquire the data. The amount of delay between acquisition and imaging for real-time operation may vary, such as a greater delay for initially locating planes of a multi-planar reconstruction with less delay for subsequent imaging. In alternative embodiments, the ultrasound data is stored in the memory 14 from a previous imaging session and used for generating the multi-planar reconstruction without concurrent acquisition.

The memory 14 is additionally or alternatively a computer readable storage medium with processing instructions. The memory 14 stores data representing instructions executable by the programmed processor 12 for multi-planar reconstruction for ultrasound volume data. The instructions for implementing the processes, methods and/or techniques discussed herein are provided on computer-readable storage media or memories, such as a cache, buffer, RAM, removable media, hard drive or other computer readable storage media. Computer readable storage media include various types of volatile and nonvolatile storage media. The functions, acts or tasks illustrated in the figures or described herein are executed in response to one or more sets of instructions stored in or on computer readable storage media. The functions, acts or tasks are independent of the particular type of instructions set, storage media, processor or processing strategy and may be performed by software, hardware, integrated circuits, firmware, micro code and the like, operating alone or in combination. Likewise, processing strategies may include multiprocessing, multitasking, parallel processing and the like. In one embodiment, the instructions are stored on a removable media device for reading by local or remote systems. In other embodiments, the instructions are stored in a remote location for transfer through a computer network or over telephone lines. In yet other embodiments, the instructions are stored within a given computer, CPU, GPU, or system.

The processor 12 is a general processor, digital signal processor, three-dimensional data processor, graphics processing unit, application specific integrated circuit, field programmable gate array, digital circuit, analog circuit, combinations thereof, or other now known or later developed device for processing medical image data. The processor 12 is a single device, a plurality of devices, or a network. For more than one device, parallel or sequential division of processing may be used. Different devices making up the processor 12 may perform different functions, such as a scanning controller and an image generator operating separately. In one embodiment, the processor 12 is a control processor or other processor of a medical diagnostic imaging system, such as a medical diagnostic ultrasound imaging system processor. The processor 12 operates pursuant to stored instructions to perform various acts described herein, such as obtaining data, deriving anatomical information, setting an imaging parameter and/or controlling imaging.

In one embodiment, the processor 12 receives acquired ultrasound data during scanning and determines locations of planes for a multi-planar reconstruction relative to the volume represented by the data. The processor 12 performs or controls other components to perform the methods described herein. The acts of the methods may be implemented by programs and/or a classifier. Any classifier may be applied, such as a model based classifier or a learned classifier or classifier based on machine learning. For learned classifiers, binary or multi-class classifiers may be used, such as Bayesian or neural network classifiers. In one embodiment, a multi-class boosting classifier with a tree and cascade structure is used. The classifier is instructions, a matrix, a learned code, or other software and/or hardware for distinguishing between information in a medical image. Learned feature vectors are used to classify the anatomy. For example, the classifier identifies a canonical view, tissue structure, flow pattern, or combinations thereof from ultrasound data. In cardiac imaging, the classifier may identify cardiac structure associated with a particular view of a heart. The view is a common or standard view (e.g., apical four chamber, apical two chamber, left parasternal, or sub-coastal), but other views may be recognized. The cardiac structure is the heart walls or other structure defining the view or a structure associated with the view. For example, a valve associated with an apical four chamber view is identified.

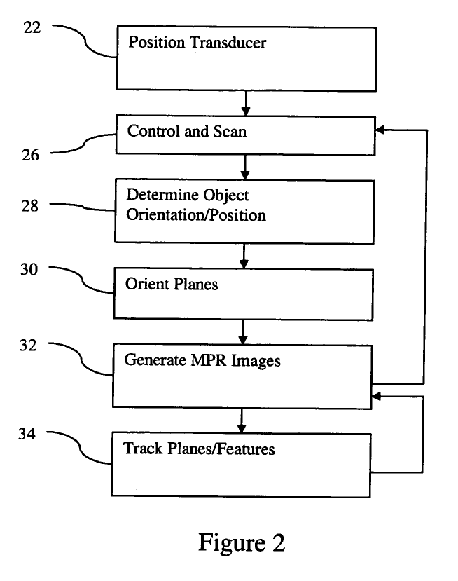

FIG. 2 shows a method for multi-planar reconstruction for ultrasound volume data. The method is implemented by a medical diagnostic imaging system, a review station, a workstation, a computer, a PACS station, a server, combinations thereof, or other device for image processing medical ultrasound data. For example, the system or computer readable media shown in FIG. 1 implements the method, but other systems may be used. The method is implemented in the order shown or a different order. Additional, different, or fewer acts may be performed. For example, acts 22 and/or 34 are optional. As another example, scanning is performed in act 26 without controlling the scan. The feedback from act 32 to act 26 may not be provided or may feedback to a different act and/or from a different act.

The acts 22-34 are performed in real-time, such as during scanning in act 26. The user may view images of act 32 while scanning in act 26. The images may be associated with previous performance of acts 22-30 in the same imaging session, but with different volume data. For example, acts 22-32 are performed for an initial scan. Acts 22, 26, 34 and 32 are performed for subsequent scans during the same imaging session. The scan of act 26 may result in images from act 32 in a fraction of a second or longer time period (e.g., seconds) and still be real-time with the scanning. The user is provided with imaging information representing portions of the volume being scanned while scanning.

In act 22, an ultrasound transducer is positioned adjacent, on or within a patient. A volume scanning transducer is positioned, such as a wobbler or multi-dimensional array. For adjacent or on a patient, the transducer is positioned directly on the skin or acoustically coupled to the skin of the patient. For within the patient, an intraoperative, intercavity, catheter, transesophageal, or other transducer positionable within the patient is used to scan from within the patient.

The user may manually position the transducer, such as using a handheld probe or manipulating steering wires. Alternatively, a robotic or mechanical mechanism positions the transducer.

In act 26, the acquisition of data by scanning a volume region of a patient is controlled. Transmit and receive scanning parameters are set, such as loading a sequence of transmit and receive events to sequentially scan a volume. The transmit and receive beamformers are controlled to acquire ultrasound data representing a volume of the patient adjacent to the transducer.

In response to the control, the volume region of the patient is scanned in act 26. The wobbler or multi-dimensional array generates acoustic energy and receives responsive echoes. In alternative embodiments, a one-dimensional array is manually moved for scanning a volume.

One or more sets of data are obtained. The ultrasound data corresponds to a displayed image (e.g., detected and scan converted ultrasound data), beamformed data, detected data, and/or scan converted data. The ultrasound data represents a region of a patient. The region includes tissue, fluid or other structures. Different structures or types of structures react to the ultrasound differently. For example, heart muscle tissue moves, but slowly as compared to fluid. The temporal reaction may result in different velocity or flow data. The shape of a structure or spatial aspect may be reflected in B-mode data. One or more objects, such as the heart, an organ, a vessel, fluid chamber, clot, lesion, muscle, and/or tissue are within the region. The data represents the region.

For example, FIG. 3 shows a volume region 40 with an object 42 at least partly within the region 40. The object 42 may have any orientation within the volume region 40. The position of planes 44 relative to the object 42 is determined for multi-planar reconstruction.

Referring to FIGS. 2 and 3, a processor determines from the ultrasound data responsive to the scanning an orientation of the object 42 within the volume region 40 or relative to the transducer in act 28. The determination is made while scanning in act 26. The data used for the determination is previously acquired, such as an immediately previous scan, or data presently being acquired.

The orientation may be determined using any now known or later developed process. The processor identifies the object without user input. Alternatively, the orientation may be based, in part, on user input. For example, the user indicates the type of organs or object of interest (e.g., selecting cardiology or echocardiography imaging).

The location of features is used to determine object orientation. For example, template modeling or matching is used to identify a structure or different structures, such as taught in U.S. Pat. No. 7,092,749, the disclosure of which is incorporated herein by reference. A template is matched to a structure. In one embodiment, the template is matched to an overall feature, such as the heart tissue and chambers associated with a standard view. The template may be annotated to identify other features based on the matched view, such as identifying specific chambers or valves.

Trained classifiers may be used. Anatomical information is derived from the ultrasound data. The anatomical information is derived from a single set or a sequence of sets. For example, the shape, the position of tissue over time, flow pattern, or other characteristic may indicate anatomical information. Anatomical information includes views, organs, structure, patterns, tissue type, or other information. A feature is any anatomical structure. For cardiac imaging, the anatomical features may be a valve annulus, an apex, chamber, valve, valve flow, or other structure. Features corresponding to a combination of different structures may be used.

In one embodiment, the locations of one or more features of the object are determined by applying a classifier. For example, any of the methods disclosed in U.S. Published Patent Application No. ______ (Attorney Docket No. 2006P14951US), the disclosure of which is incorporated herein by reference, is used. The anatomical information is derived by applying a classifier. Any now known or later developed classifier for extracting anatomical information from ultrasound data may be used, such as a single class or binary classifier, collection of different classifiers, cascaded classifiers, hierarchal classifier, multi-class classifier, model based classifier, classifier based on machine learning, or combinations thereof. The classifier is trained from a training data set using a computer. Multi-class classifiers include CART, K-nearest neighbors, neural network (e.g., multi-layer perceptron), mixture models, or others. The AdaBoost.MH algorithm may be used as a multi-class boosting algorithm where no conversion from multi-class to binary is necessary. Error-correcting output code (EOCC) may be used.

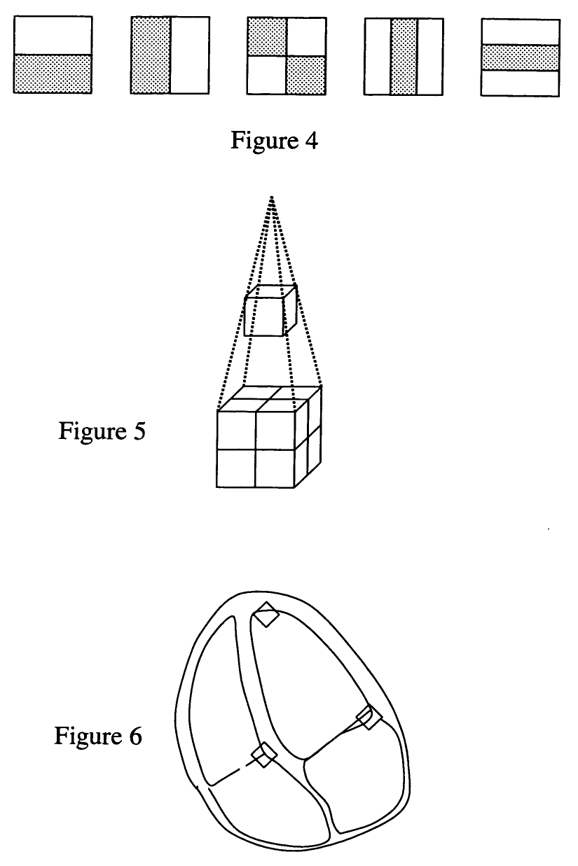

For learning-based approaches, the classifier is taught to detect objects or information associated with anatomy. For example, the AdaBoost algorithm selectively combines into a strong committee of weak learners based on Haar-like local rectangle filters whose rapid computation is enabled by the use of an integral image. FIG. 4 shows five example filters for locating or highlighting edges. A cascade structure may deal with rare event detection. The FloatBoost, a variant of the AdaBoost, may address multiview detection. Multiple objects may be dealt with by training a multi-class classifier with the cascade structure. The classifier learns various feature vectors for distinguishing between classes of features. A probabilistic boosting tree (PBT), which unifies classification, recognition, and clustering into one treatment, may be used.

A tree structure may be learned and may offer efficiency in both training and application. Often, in the midst of boosting a multi-class classifier, one class (or several classes) has been completely separated from the remaining ones and further boosting yields no additional improvement in terms of the classification accuracy. For efficient training, a tree structure is trained. To take advantage of this fact, a tree structure is trained by focusing on the remaining classes to improve learning efficiency. Posterior probabilities or known distributions may be computed, such as by correlating anterior probabilities together.

To handle the background classes with many examples, a cascade training procedure may be used. A cascade of boosted multi-class strong classifiers may result. The cascade of classifiers provides a unified algorithm able to detect and classify multiple objects while rejecting the background classes. The cascade structure corresponds to a degenerate decision tree. Such a scenario presents an unbalanced nature of data samples. The background class has voluminous samples because all data points not belonging to the object classes belong to the background class. To examine more background examples, only those background examples that pass the early stages of the cascade are used for training a current stage.

The trained classifier is applied to the ultrasound data. The ultrasound data is processed to provide the inputs to the classifier. For example, the filters of FIG. 4 are applied to the ultrasound data. For each spatial location, a matrix or vector representing the outputs of the filters is input to the classifier. The classifier identifies features based on the inputs.

The anatomical information is encoded based on a template. The image is searched based on the template information, localizing the chambers or other structure. Given a sequence of medical images, a search from the left-top corner to the right-bottom corner is performed by changing the width, height, and angle of a template box. The search is performed in a pyramid structure, with a coarse search on a lower resolution or decimated image and a refined search on a higher resolution image based on the results of the coarse search. This exhaustive search approach may yield multiple results of detections and classifications, especially around the correct view location. The search identifies relevant features. FIG. 3 shows a plurality of features 46. FIG. 6 shows an A4C view with three identified features.

In act 30, a multi-planar reconstruction is oriented as a function of the orientation of the object. The orientation of the object may be represented by two or more features. By identifying the location of features of the object, the position of the object relative to the volume region 40 and/or the transducer is determined.

Two or more planes 44 are oriented within the volume region 40 as a function of the locations of the features. Each of the planes is different, such as being defined by different features. A plane 44 may be oriented based on identification of a single feature, such as a view. Alternatively, the plane 44 is oriented based on the identification of a linear feature and a point feature or three or more point features. Any combination of features defining a plane 44 may be used. For example, an apex, four chambers, and a valve annulus define an apical four-chamber view. The apex, two chambers and a valve annulus define an apical two-chamber view in the same volume region 40.

The planes are oriented independent of an orientation of the ultrasound transducer relative to the object 42. Since the search for features is performed in three-dimensional space, the features may be identified without consideration of the orientation of the transducer. The transducer does not need to be moved to define a view or establish a plane. The planes are found based on the ultrasound data representing the volume. Orienting is performed without fixing a view relative to the transducer and by fixing the view relative to the object. Since the orientation is performed during the control of the acquisition by scanning, the user may, but is not required to, precisely position the transducer.

The features and corresponding plane orientations identify a standard or predetermined view. One or more of the planes 44 of the reconstruction are positioned relative to the object 42 such that the planes correspond to the standard and/or predetermined view. Standard views may be standard for the medical community or standard for an institution. For example in cardiac imaging, the object is a heart, and the planes are positioned to provide an apical two chamber, apical four chamber, parasternal long axis, and/or parasternal short axis view. Predetermined views include non-standard views, such as a pre-defined view for clinical testing.

One, two, or more planes are oriented to provide different views. In one embodiment, three, four or more planes are oriented, such as for echocardiography. In another embodiment, three planes (e.g., two longitudinal views at different angles of rotation about a longitudinal axis and one cross-sectional view) are oriented relative to three dimensions along a flow direction of a vessel. One or more planes may be for non-standard and/or non-predetermined views. Each plane is independently oriented based on features. Alternatively or additionally, the orientation of one plane may be used to determine an orientation of another plane.

In act 32, multi-planar reconstruction images of the object are generated from the ultrasound data. The planes define the data to be used for imaging. Data associated with locations intersecting each plane or adjacent to each plane is used to generate a two-dimensional image. Data may be interpolated to provide spatial alignment to the plane, or a nearest neighbor selection may be used. The resulting images are generated as a function of the orientation of the multi-planar reconstruction and provide the desired view. The images represent different planes 44 through the volume region 40.

In one embodiment, specific views are generated. All or a sub-set of the specific views are generated. Where planes corresponding to the views are identified, the views may be provided. For example, all the available standard or predetermined views in ultrasound data representing a region are provided. The images for each view may be labeled (e.g., A4C) and/or annotated (e.g., valve highlighted). Fewer than all available views may be provided, such as displaying no more than three views and having a priority list of views.

The images are generated during the control of the acquisition by scanning. Real-time generation allows a sonographer to verify the desired information or images are obtained.

FIG. 2 shows a feedback for repeating the determining act 28, orienting act 30, and generating act 32 a plurality of times while scanning (act 26) in a same imaging session. The generating of act 32 may be performed more frequently, such as applying previously determined plane positions to subsequent data sets. The determining and orienting acts 28, 30 may be performed less frequently. Alternatively, the determining and orienting acts 28, 30 are performed for each set of ultrasound data representing the volume region.

The repetition of the determining and orienting acts 28, 30 may be periodic. Every set, second set, third set or other number of sets of data acquired, the acts are triggered. The trigger may be based on timing, a clock, or a count. Other trigger events may be used, such as heart cycle events, detected transducer motion, detected object motion, or failure of tracking.

As an alternative or in addition to repeating the determining and orienting acts 28, 30, the features or views are tracked between sets of data and the plane positions are refined based on the tracking. The anatomical view planes of the multi-planar reconstruction are tracked as a function of time. For example, speckle or feature tracking (e.g., correlation or minimum sum of absolute differences) of the features is performed as a function of time. As another example, velocity estimates are used to indicate displacement. Other tracking techniques may be used. The amount of displacement, direction of displacement, and/or rotation of the features or two-dimensional image region defining a plane is determined. The plane position is adjusted based on the tracking. Images are generated in act 32 using the adjusted planes.

One example embodiment for determining an orientation of planes in a multi-planar reconstruction uses a pyramid of ultrasound data sets. More rapid determination may be provided. The ultrasound data is used to create two or more sets of data with different resolution (see FIG. 5 for a graphical example). For example, a one set of data has fine resolution, such as the scan resolution, and another set of data has a coarse resolution, such as the fine set decimated by ½ in each dimension. The sets represent the same object in the same volume. Three or more sets may be used.

The sets are in any format, such as Cartesian or polar coordinate. In one embodiment, the ultrasound data is acquired in an acoustic (e.g., polar) coordinate format, and the Cartesian or display space is populated in real time with only visible surfaces or selected planes. In another embodiment using a scan converter, processor, or graphics processing unit, real-time conversion from the acoustic space to the Cartesian or display space is feasible. The ultrasound data is processed in the Cartesian space (e.g., 3D grid) to orient the multi-planar reconstruction.

Given a volumetric pyramid, a hierarchical coarse-to-fine strategy is implemented. The data is processed on the highest level, such as scanning for feature points and/or planes. The subsequent levels are processed for further details. The pyramid processing may improve the processing speed. Alternatively, a single set of ultrasound data is used without the data pyramid.

The locations of multiple planes are detected. The 3D volumetric data is stored in the spherical coordinates. The relationship between the Cartesian coordinates (x, y, s) and the spherical coordinates (r, θ, Φ) is as follows:

x=r sin Φ sin θ; y=r sin Φ cos θ; z=r cos Φ

One parameterization of a plane in the Cartesian coordinates is:

x sin Φ₀ sin θ₀+y sin Φ₀ cos θ₀+z cos Φ₀=r₀

where (r₀, θ₀, Φ₀) is the spherical coordinates of the perpendicular foot of the origin to the plane. Therefore, the plane in the spherical coordinates is parameterized as:

r sin Φ sin Φ₀ cos (θ−θ₀)+r cos Φ cos Φ₀=r₀

In a first stage, an estimate of the multiple planes is performed. Using the volume pyramid in the Cartesian space, the data is processed in a hierarchical manner to detect reliable feature points, such as an apex, valve annulus points, and/or chambers (e.g., the chamber of the left ventricle). The features provide a rough estimate of the positions of the multiple planes. Using the features, the whole heart or structure may be aligned to a canonical model.

Object features are identified. A processor identifies a plurality of features of the object from the first coarse set of ultrasound data. Larger features may be used for determining the locations in the coarse set. The features indicate the likely location of smaller features. The smaller features are then identified in the fine set of the volume pyramid based on the larger features. Alternatively, the smaller features are identified regardless of the larger features. The location of the larger features may be refined using the fine set with or without identifying other features in the fine set.

The processor determines locations of planes for multi-planar reconstruction as a function of the features of the object. As discussed above for act 30, the position of planes of the multi-planar reconstruction is located as a function of the object features. Images may then be generated.

One embodiment is for multi-plane detection and tracking in real-time 3D echocardiography. In this embodiment, the location for the multiple planes is initially determined without required user adjustment of an imaging or scanning plane. Once the positions are determined, the planes are tracked for subsequent imaging using a different approach.

In a second stage, detection for refinement may be programmed. In one embodiment, a learning approach to detection is used. An annotated database is obtained. Training positive and negative examples are extracted from the database.

A refined estimate of the positions of the multiple planes is determined from the set of data with a finer resolution. In the Cartesian space, the estimates of each plane positioning are refined. Any now known or later developed refinement may be used. In two possible embodiments, a designed object detector that discriminates the subtle difference in the appearance is trained, or a regressor is learned to infer the estimate. Alternatively, projection pursuit is used.

Points like the apex and valve annulus present distinct structures that may be well described by specified features. In addition, the heart chambers have semantic constraints that could be learned by higher-level classifiers. A hierarchical representation, modeling, and detection of the heart structures may be used.

In one approach, plane-specific detectors are learned. One detector for one particular plane may be learned. For example, an A4C detector and an A2C detector are learned separately. In another approach, a joint detector for multiple planes that are treated as an entity is learned.

The detector is a binary classifier F(I):
if F(I)≧0, then I is positive;
   else, I is negative.
      The function F( I) also gives a confidence of being positive. The higher F(I) is, the more confident the input I is positive. The confidence may be interpreted as a posterior probability:

$$p ( 1 ) = \frac{exp ( F ( I ) )}{exp ( F ( I ) ) + exp ( - F ( I ) )}$$

The binary classifier known as the AdaBoosting algorithm, probabilistic boosting tree (PDT) or other classifiers may be used. With the ultrasound data, the boosting algorithm is used to select Haar features, which are amenable to rapid evaluation. The Haar features are combined to form a committee classifier. An image-based multi-class classifier to further differentiate multiple planes may be used in addition.

Given an ultrasound volume V and a configuration a, multiple planes denoted by I=V(a) are extracted. The optimal parameter a that maximizes the detector classifier p(I) is searched for where:

$${α}^{⋒} = arg {max}_{α} p ( V ( α ) )$$

When separate plane-specific classifiers are learned, the overall detector classifier p(I) is simply p(I)=p1(I)*p2(I)* . . . *pn(I), that is, a product of n quantities. Since the multiple planes of interest are geometrically constrained, some degrees of freedom are provided when performing the exhaustive search.

In projection pursuit, the posterior probability p(V(a)) is replaced by a pursuit cost function. For example, it can be based on projected histogram or other measures.

Regression may be used for refinement with the fine set of ultrasound data. One practical consideration is the searching speed especially when confronting the three-dimensional parameter space. To accelerate the speed, a function:

δα=g(I).

is learned, where δα is the difference between the current parameter α and the ground truth \acute{α}, as represented by:

δα=α−\ddot{α}

In one embodiment, the locations of the planes are refined. The processor uses any function to refine the locations, such as a classifier, regressor, or combinations thereof. For example, from the annotated database, example pairs {(Iₙ, δαₙ)}ₙ₌₁^{N}. An image-based regression task is used to find the target function g(I) that minimizes the cost function:

$$arg {min}_{g} L ( g ) = {\sum }_{n = 1}^{N} L ( g ( {I}_{n} ) , {δα}_{n} ) + λ R ( g )$$

where L(g(lₙ), δαₙ) is the loss function and R(g) is a regularization term.

Based on the initial parameter a, the multiple slices I=V(α) are formed, and then the parameter δαₙ is inferred by applying the regression function. An updated estimate α₁=α₀+δαₙ is obtained. By iterating the above process until convergence, the parameter a may be inferred reasonably close to the ground truth. The discriminative classifier is used to further tweak the estimate.

After detecting the planes from the ultrasound data, the planes are tracked. In real-time acquisition, after locking the multiple planes, the planes are tracked. Any tracking may be used. In an alternative embodiment, the detector is applied for every scan or ultrasound data representing the volume region.

Another approach is to use multiple hypotheses tracking. In multiple hypotheses tracking, hypotheses of a, are generated and verified by the filtering probability p(α₁|V_{1:t}). Tracking is performed in real-time. Due to sampling smoothness in the temporal dimension, the parameter cannot undergo a sudden change. Assume that a obeys a Markov model:

αₜ=αₜ₋₁+uₜ

where uₜ is a noise component. If the classifier p(I) is used as a likelihood measurement q(Vₜ|αₜ)=p(Vₜ(αₜ)), the posterior filtering probability p(αₜ|V_{1:t}) is calculated. The optimal a, is estimated from the posterior filtering probability.

The Kalman filter may provide an exact solution. Often, the above time series system is not linear and the distributions are non-Gaussian. The sequential Monte Carlo (SMC) algorithm may be used to derive an approximate solution, allowing real-time tracking.

While the invention has been described above by reference to various embodiments, it should be understood that many changes and modifications can be made without departing from the scope of the invention. It is therefore intended that the foregoing detailed description be regarded as illustrative rather than limiting, and that it be understood that it is the following claims, including all equivalents, that are intended to define the spirit and scope of this invention.

I (we) claim:

1. A method for multi-planar reconstruction for ultrasound volume data, the method comprising:
positioning an ultrasound transducer adjacent, on or within a patient;
scanning a volume region with the ultrasound transducer;
determining, with a processor and from data responsive to the scanning, a first orientation of an object within the volume region while scanning;
orienting a multi-planar reconstruction as a function of the first orientation of the object and independently of a second orientation of the ultrasound transducer relative to the object; and
generating multi-planar reconstruction images of the object from the data while scanning, the images being a function of the orientation of the multi-planar reconstruction.

2. The method of claim 1 wherein positioning comprises the user positioning a wobbler or multi-dimensional array, wherein scanning comprises scanning the volume region with the wobbler or multi-dimensional array, and wherein generating the multi-planar reconstruction comprises forming at least two two-dimensional images associated with different planes in the volume region.

3. The method of claim 1 wherein determining, generating and orienting are performed in real-time with the scanning.

4. The method of claim 1 wherein orienting the multi-planar reconstruction comprises positioning at least one plane of the reconstruction relative to the object such that the at least one plane corresponds to a standard view.

5. The method of claim 4 wherein the object comprises a heart, and wherein positioning comprises positioning the at least one plane for an apical two chamber, apical four chamber, parasternal long axis, or parasternal short axis view.

6. The method of claim 1 wherein orienting comprises orienting without fixing a view relative to the transducer and by fixing the view relative to the object.

7. The method of claim 1 further comprising:
repeating the determining, orienting, and generating a plurality of times while scanning in a same imaging session.

8. The method of claim 1 wherein orienting comprises orienting a plurality of planes to a respective plurality of views for the object.

9. The method of claim 1 further comprising:
tracking anatomical view planes of the multi-planar reconstruction as a function of time.

10. The method of claim 1 wherein generating comprises generating available standard views with the processor from the data for the object.

11. The method of claim 1 wherein determining comprises identifying a plurality of features of the object from the data in a first coarse set and in a second fine set of a volume pyramid.

12. The method of claim 1 wherein determining and orienting comprise:
identifying object features;
locating planes of the multi-planar reconstruction as a function of the object features; and
refining locations of the planes with a classifier, regressor, or combinations thereof.

13. The method of claim 1 further comprising:
tracking planes of the multi-planar reconstruction with multiple hypotheses tracking.

14. In a computer readable storage medium having stored therein data representing instructions executable by a programmed processor for multi-planar reconstruction for ultrasound volume data, the storage medium comprising instructions for:
controlling acquisition by scanning a volume region of a patient;
determining, from ultrasound data responsive to the acquisition, locations of features of an object within the volume region represented by the data, the determining being during control of the acquisition by scanning;
orienting a plurality of planes within the volume region as a function of the locations of the features, the orienting being independent of an orientation of an ultrasound transducer relative to the object, each of the plurality of planes being different from the other ones of the plurality of planes; and
generating images of the object from the data for each of the planes.

15. The instructions of claim 14 wherein orienting and generating are performed during the control of the acquisition by scanning.

16. The instructions of claim 14 wherein orienting comprises positioning the planes relative to the object such that the planes correspond to a predetermined views.

17. The instructions of claim 14 further comprising:
tracking the features as a function of time.

18. The instructions of claim 14 wherein determining comprises determining from the data at different resolutions.

19. The instructions of claim 14 further comprising:
refining locations of the planes with a classifier, regressor, or combinations thereof.

20. The instructions of claim 14 further comprising:
tracking the planes with multiple hypotheses tracking.

21. A method for multi-planar reconstruction for ultrasound volume data, the method comprising:
obtaining ultrasound data in a first coarse set and a second fine set, the ultrasound data representing an object in a volume;
identifying, with a processor, a plurality of features of the object from the first coarse set of ultrasound data;
determining, with the processor, locations of planes for multi-planar reconstruction as a function of the features of the object; and
refining, with the processor, the locations as a function of the second fine set.

22. The method of claim 21 wherein refining comprises refining with a classifier, regressor, or combinations thereof.

23. The method of claim 21 further comprising:
tracking the planes with multiple hypotheses tracking.
